(19) United States
(12) Patent Application Publication
(10) Pub. No.: US 2020/0005010 A1
(43) Pub. Date: Jan. 2, 2020

(54) CELL STRUCTURE AND OPERATION METHOD FOR FINGERPRINT SENSOR EMPLOYING PSEUDO-DIRECT SCHEME

(71) Applicant: HANSHIN UNIVERSITY INDUSTRY-ACADEMIC COOPERATION FOUDATION, Osan-si, Gyeonggi-do (KR)

(72) Inventors: Hyeop Goo YEO, Hwaseong-si (KR); Seung Min JUNG, Seongnam-si (KR)

(73) Assignee: Hanshin University Industry-Academic Cooperation Foundation, Osan-si, Gyeonggi-do (KR)

(21) Appl. No.: 16/476,266

(22) PCT Filed: Nov. 23, 2017
(86) PCT No.: PCT/KR2017/013424

§ 371 Date: Sep. 16, 2019

(30) Foreign Application Priority Data
Jan. 5, 2017 (KR) 10-2017-0001915

(51) Int. Cl. G06K 9/00

(52) U.S. Cl. CPC G06K 9/0002; A61B 5/1172; G06K 9/00053

(57) ABSTRACT

A fingerprint sensor in accordance with an exemplary embodiment includes a sensor plate including a plurality of cells, a driving signal generation unit disposed between an external voltage input terminal and the sensor plate to generate a driving signal, an active output voltage feedback unit disposed between one end of a variable capacitor and a voltage output terminal, and a shielding plate provided below the sensor plate and connected to an output terminal of the active output voltage feedback unit to block a noise generated from a circuit disposed below the sensor plate. Here, the fingerprint sensor further includes a driving signal control switch having one end connected to the driving signal generation unit and the other end connected to each of the plurality of cells of the sensor plate to set at least a portion of the plurality of cells of the sensor plate as a sensing cell and at least a portion of cells disposed around the set sensing cells as a driving cell.

### TECHNICAL FIELD

The present disclosure relates to a cell structure and a method of a fingerprint sensor using a pseudo-direct method.

More particularly, the present disclosure relates to a cell structure and a driving method of a fingerprint sensor using a pseudo-direct method, which acquires a fingerprint image by applying a driving signal to a fingerprint sensor without a bezel.

### BACKGROUND ART

In recent years, demand for biometric sensors has been increasing. Particularly, as a fingerprint is used for personal identification and payment in a mobile field, demand for fingerprint sensors has been rapidly increasing.

In case of the fingerprint sensor used in the mobile field, a capacitive fingerprint sensor, which is cheap and occupies a small volume, is used.

In order to drive the capacitive fingerprint sensor in an optimized state, a method for directly applying a driving signal to a finger using a bezel has been used.

However, the method for directly applying the driving signal to the finger using the bezel has a limitation in design aspect and causes increase in system costs due to the bezel.

In recent years, in order to resolve the limitation generated by using the bezel, a capacitive fingerprint sensor technology without the bezel has been developed.

However, the capacitive fingerprint sensor without the bezel exhibits a performance lower than that of a typical fingerprint sensor without the bezel.

### DISCLOSURE OF THE INVENTION

### Technical Problem

The present disclosure provides a cell structure and a driving method of a fingerprint sensor using a pseudo-direct method without a bezel, which provides an equivalent performance to that of a typical capacitive fingerprint sensor having a bezel.

### Technical Solution

In accordance with an exemplary embodiment, a fingerprint sensor includes: a sensor plate including a plurality of cells; a driving signal generation unit disposed between an external voltage input terminal and the sensor plate to generate a driving signal; an active output voltage feedback unit disposed between one end of a variable capacitor and a voltage output terminal; and a shielding plate provided below the sensor plate and connected to an output terminal of the active output voltage feedback unit to block a noise generated from a circuit disposed below the sensor plate. Here, the fingerprint sensor further includes a driving signal control switch having one end connected to the driving signal generation unit and the other end connected to each of the plurality of cells of the sensor plate to set at least a portion of the plurality of cells of the sensor plate as a sensing cell and at least a portion of cells disposed around the set sensing cells as a driving cell. Also, a driving signal generated from the driving signal generation unit is provided to the set driving cell through the driving signal control switch, and the set sensing cells sense a fingerprint signal.

The plurality of cells of the sensor plate may be set: as the driving cell when the driving signal control switch is in an on-state; and as the sensing cell when the driving signal control switch is in an off-state, and the driving cell may be disposed adjacent to the sensing cell.

In accordance with another exemplary embodiment, a fingerprint sensor includes: a sensor plate including a plurality of cells; a driving signal generation unit disposed between an external voltage input terminal and the sensor plate to generate a driving signal; an active output voltage feedback unit disposed between one end of a variable capacitor and a voltage output terminal; and a shielding plate provided below the sensor plate and connected to an output terminal of the active output voltage feedback unit to block a noise generated from a circuit disposed below the sensor plate. Here, the fingerprint sensor further includes: a driving signal control switch having one end connected to the driving signal generation unit and the other end connected to the sensor plate to set at least a portion of the plurality of cells of the sensor plate as a sensing cell and at least a portion of cells, which are spaced by a predetermined area from the set sensing cells, as a driving cell; and an isolation cell control switch having one end connected to the driving signal control switch and the other end connected to the ground to set cells in a predetermined area, in which the sensing cell and the driving cell are spaced apart from each other, as an isolation cell. Also, a driving signal generated from the driving signal generation unit is provided to the set driving cell through the driving signal control switch, the set sensing cells sense a fingerprint signal, and the set isolation cell is connected to the ground.

The plurality of cells of the sensor plate may operate: as the driving cell when the driving signal control switch is in a on-state, and the isolation cell control switch is in an off-state; as the sensing cell when all of the driving signal control switch and the isolation cell control switch are in the off-state; and as the isolation cell when the driving signal control switch is in the off-state, and the isolation cell control switch is in an on-state, and the isolation cell may be provided between the driving cell and the sensing cell.

The active output voltage feedback unit may include: a buffer circuit part configured to feedback an output terminal of an operational amplifier to an inverting input terminal of the operational amplifier; a first switch and a second switch, which are parallel-connected to the other end of the driving signal control switch and an isolation cell control switch, respectively; a storage capacitor having one end connected between one end of the first switch and a non-inverting input terminal of the operational amplifier of the buffer circuit part and the other end connected to the ground; and a reset switch having one end connected to the one end of the first switch, the non-inverting input terminal of the operational amplifier of the buffer circuit part, and one end of the storage capacitor and the other end connected to the ground.

In accordance with yet another exemplary embodiment, a method for sensing a fingerprint includes: setting at least a portion of a plurality of cells of a sensor plate as a sensing cell configured to measure a capacitance formed between a finger and the sensing plate; setting at least a portion of cells around the set sensing cells of the plurality of cells of the sensor plate as a driving cell configured to provide a driving signal to a finger; providing a driving signal from the driving cell to the finger; and outputting a sensing signal by integrating a capacitance detected in the sensing cell by the signal applied from the driving cell to the finger.

The setting of the sensing cell may turn-off a driving signal control switch to set a portion of the plurality of cells of the sensor plate as the sensing cell, and the setting of the driving cell may turn-on the driving signal control switch to set cells, which are not set as the sensing cell, of the cells of the sensor plate as the driving cell.

In accordance with still another exemplary embodiment, a method for sensing a fingerprint includes: setting at least a portion of a plurality of cells of a sensor plate as a sensing cell configured to measure a capacitance formed between a finger and the sensing plate; setting at least a portion of cells spaced by a predetermined area from the set sensing cells as a driving cell configured to provide a driving signal to a finger; setting cells in a predetermined area in which the sensing cell and the driving cell are spaced apart from each other as an isolation cell; providing a driving signal to the driving cell; and outputting a sensing signal by integrating a capacitance detected in the sensing cell by the signal applied from the driving cell to the finger.

The setting of the sensing cell may turn-off all of a driving signal control switch and an isolation cell control switch to set a portion of the plurality of cells of the sensor plate as the sensing cell, the setting of the isolation cell may turn-off the driving signal control switch and turn-on the isolation cell control switch to set a portion of cells, which are adjacent to the set sensing cells of the plurality of cells of the sensor plate, as the isolation cell, and the setting of the driving cell may turn-on the driving signal control switch and turn-off the isolation cell control switch to set cells, which are not set as the sensing cell and the isolation cell, of the plurality of cells of the sensor plate as the driving cell.

### Advantageous Effects

The present disclosure may provide the equivalent performance to that of the capacitive fingerprint sensor having the bezel although the bezel is not contained.

Also, the present disclosure may improve in entire design of the product in which the fingerprint sensor is contained by realizing the capacitive fingerprint sensor without the bezel.

### BRIEF DESCRIPTION OF THE DRAWINGS

FIG. 1 is a configuration view illustrating a fingerprint sensor using a pseudo-direct method in accordance with an exemplary embodiment.

FIG. 2 is a plane view illustrating the fingerprint sensor using a pseudo-direct method in accordance with an exemplary embodiment.

FIG. 3 is a side view illustrating the fingerprint sensor using a pseudo-direct method in accordance with an exemplary embodiment.

FIG. 4 is a configuration view illustrating a fingerprint sensor using a pseudo-direct method in accordance with another exemplary embodiment.

FIG. 5 is a plan view illustrating the fingerprint sensor using a pseudo-direct method in accordance with another exemplary embodiment.

FIG. 6 is a side view illustrating the fingerprint sensor using a pseudo-direct method in accordance with another exemplary embodiment.

### MODE FOR CARRYING OUT THE INVENTION

Hereinafter, with reference to the accompanying drawings, embodiments of the present invention will be described in detail for those of ordinary skilled in the art to be able to perform. The invention may, however, be embodied in many different forms and should not be construed as being limited to the embodiments set forth herein. In the drawings, anything unnecessary for describing the present disclosure will be omitted for clarity, and also like reference numerals in the drawings denote like elements.

It will be understood that although the terms of first and second are used herein to describe various elements, these elements should not be limited by these terms. Terms are only used to distinguish one component from other components. For example, a first element referred to as a first element in one embodiment can be referred to as a second element in another embodiment without departing from the scope of the appended claims. In the following description, the technical terms are used only for explaining a specific exemplary embodiment while not limiting the present disclosure. The terms of a singular form may include plural forms unless referred to the contrary.

In this specification below, when one part is referred to as being ‘connected’ to another part, it should be understood that the former can be ‘directly connected’ to the latter, or ‘electrically connected to the latter via an intervening part. Furthermore, when it is described that one comprises (or includes or has) some elements, it should be understood that it may comprise (or include or has) only those elements, or it may comprise (or include or have) other elements as well as those elements if there is no specific limitation. In the present specification, “step of ˜(ing)” or “step of” does not mean “step for”.

Meanwhile, for the terms used in the present disclosure, general terms widely currently used have been selected as possible as they can. In a specific case, terms arbitrarily selected by an applicant may be used. In this case, since the meaning thereof is described in detail in the detailed description of the specification, the present disclosure should be understood in an aspect of meaning of such terms, not the simple names of such terms. It will be understood that although the terms first and second are used herein to describe various elements, these elements should not be limited by these terms.

1. A structure of a fingerprint sensor using a pseudo-direct method in accordance with an exemplary embodiment

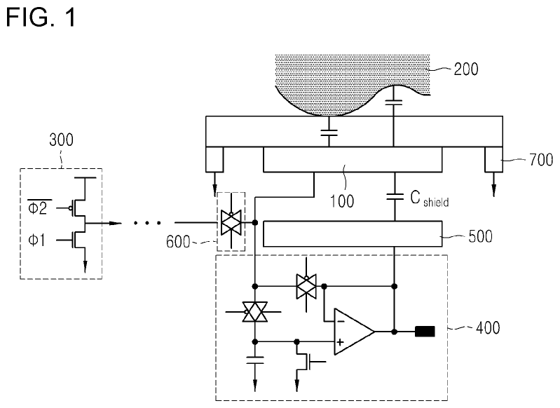

FIG. 1 is a configuration view illustrating a fingerprint sensor using a pseudo-direct method in accordance with an exemplary embodiment.

Referring to FIG. 1, a fingerprint sensor using a pseudo-direct method in accordance with an exemplary embodiment may include: a sensor plate 100 including a plurality of cells; a variable capacitor provided between a finger 200 and the sensor plate 100; a driving signal generation unit 300 disposed between an external voltage input terminal and the sensor plate 100 to generate a driving signal; an active output voltage feedback unit 400 disposed between one end of the variable capacitor and a voltage output terminal; a shielding plate 500 provided below the sensor plate 100 and connected to an output terminal of the active output voltage feedback unit 400 to block a noise generated from a circuit disposed below the sensor plate 100; and a driving signal control switch 600 having one end connected to the driving signal generation unit 300 and the other end connected to each of the plurality of cells of the sensor plate 100 and setting a portion of the plurality of cells of the sensor plate 100 as the sensing cell 110 or the driving cell 120.

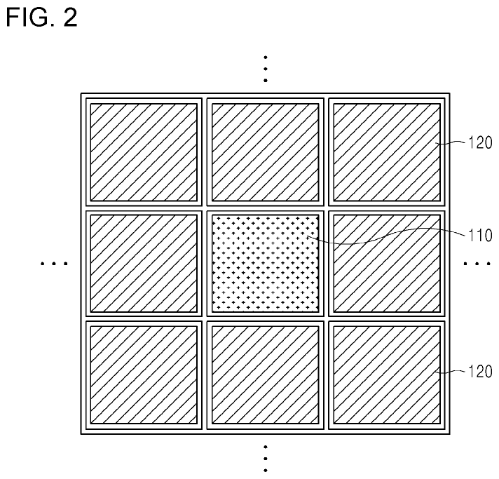

FIG. 2 is a top view illustrating the fingerprint sensor using the pseudo-direct method in accordance with an exemplary embodiment.

Referring to FIG. 2, a fingerprint sensor using a pseudo-direct method in accordance with an exemplary embodiment may set a cell disposed at a center of 3×3 areas as a sensing cell 110 and the rest neighboring eight cells as a driving cell 120.

When the sensing cell 110 and the driving cell 120 are set as described above, a limitation of weak detection of a capacitance in case of a cell that is disposed away from a portion providing a driving signal in a typical bezel-less structure may be resolved.

Although only one sensing cell 110 is described in the exemplary embodiment, two or more plurality of cells, which are adjacent to each other, may be set as the sensing cell 110.

A method for setting the plurality of cells of the sensor plate 100 as the sensing cell 110 or the driving cell 120 will be described below.

When the driving signal control switch 600 of the sensor plate 100, which is connected to an arbitrary cell, is in an on-state, as a driving signal generated from the driving signal generation unit 300 is provided to the cell, the cell may operate as the driving cell 120, and a cell connected to the driving signal control switch 600 in an off-state may operate as the sensing cell 110.

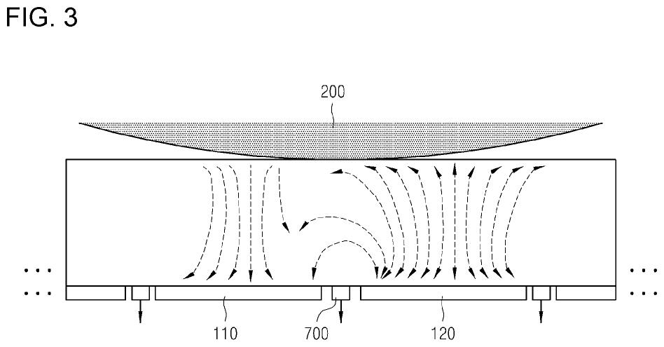

FIG. 3 is a side view illustrating the fingerprint sensor using the pseudo-direct method in accordance with an exemplary embodiment in FIG. 2.

Referring to FIG. 3, a feature, in which a driving signal is provided to the finger 200 from the cell, which is set as the driving cell 120, of the plurality of cells of the sensor plate 100, and a capacitance is measured in the sensing cell 110, is illustrated.

Although most of the driving signal provided from the driving cell 120 is applied to the finger 200 in the above-described structure, a portion of the driving signal may be introduced to the sensing cell 110 without passing the finger from the driving cell 120.

Thus, the driving cell 120 and the sensing cell 110 may be spaced by a predetermined distance from each other in order to reduce an error that is detected when the driving signal applied from the driving cell 120 is directly transmitted to the sensing cell 110 without passing the finger.

For example, the spaced predetermined distance may be a distance between the plurality of cells of the sensor plate 100. The spaced predetermined distance may serve as the ground, or a separate ground area 700 may be provided.

Also, as a dielectric layer provided at an upper portion of the sensor plate 100 increases in thickness, the driving signal applied from the adjacent driving cell 120 and directly transmitted to the sensing cell 110 without passing the finger 200 may increase in magnitude. In this case, the distance between the driving cell 120 and the sensing cell 110 is required to increase.

Also, the active output voltage feedback unit 400 may include: a buffer circuit part that feedbacks an output terminal of an operational amplifier to an inverting input terminal of the operational amplifier; a first switch and a second switch, which are parallel-connected to the other end of the driving signal control switch; a storage capacitor having one end connected between one end of the first switch and a non-inverting input terminal of the operational amplifier of the buffer circuit part and the other end connected to the ground; and a reset switch having one end connected to the one end of the first switch, the non-inverting input terminal of the operational amplifier of the buffer circuit part, and one end of the storage capacitor and the other end connected to the ground.

More particularly, as the buffer circuit part operates as a voltage follower, a parasitic capacitor C_{shield} may be charged so that the input terminals of the buffer circuit have the same voltage as each other. Thus, the parasitic capacitor C_{shield} may be removed.

Also, the active output voltage feedback unit 400 may integrate a capacitance detected by the sensing cell 120 to the storage capacitor and output the integrated capacitance to the output terminal of the active output voltage feedback unit.

2. A fingerprint sensor using a pseudo-direct method in accordance with another exemplary embodiment

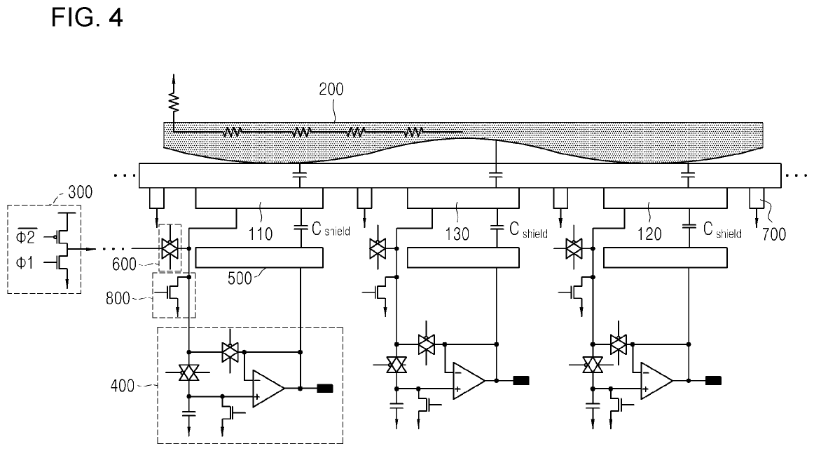

FIG. 4 is a configuration view illustrating a fingerprint sensor using a pseudo-direct method in accordance with another exemplary embodiment.

Referring to FIG. 4, the fingerprint sensor using a pseudo-direct method in accordance with another exemplary embodiment may include: a sensor plate 110, 120, and 130 including a plurality of cells; a variable capacitor provided between a finger 200 and the sensor plate 110, 120, and 130; a driving signal generation unit 300 disposed between an external voltage input terminal and the sensor plate 110, 120, and 130 to generate a driving signal; an active output voltage feedback unit 400 disposed between one end of the variable capacitor and a voltage output terminal; a shielding plate 500 provided below the sensor plate 110, 120, and 130 and connected to an output terminal of the active output voltage feedback unit 400 to block a noise generated from a circuit disposed below the sensor plate 110, 120, and 130; a driving signal control switch 600 having one end connected to the driving signal generation unit 300 and the other end connected to each of the plurality of cells of the sensor plate 110, 120, and 130 and setting a portion of the plurality of cells of the sensor plate 110, 120, and 130 as the sensing cell 110 or the driving cell 120; and an isolation cell control switch 800 having one end connected to the driving signal control switch 600 and the other end connected to the ground to set a portion of the censing cell 110 as an isolation cell 130.

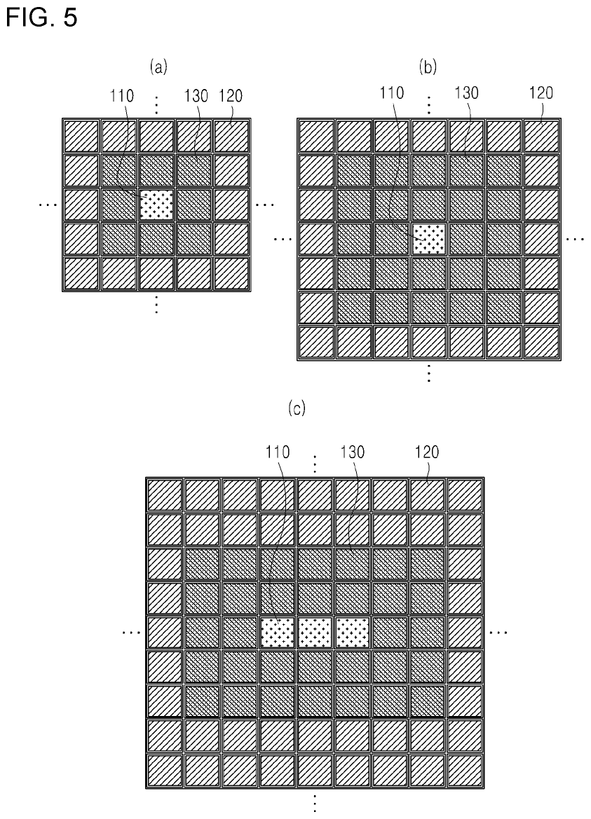

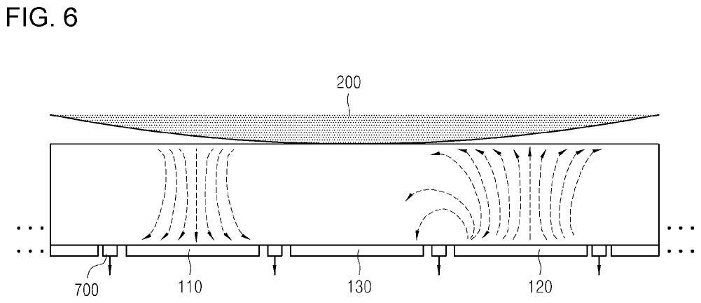

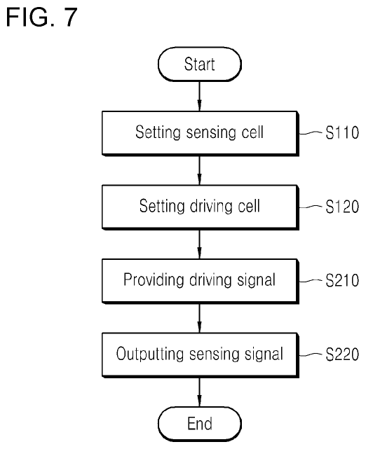

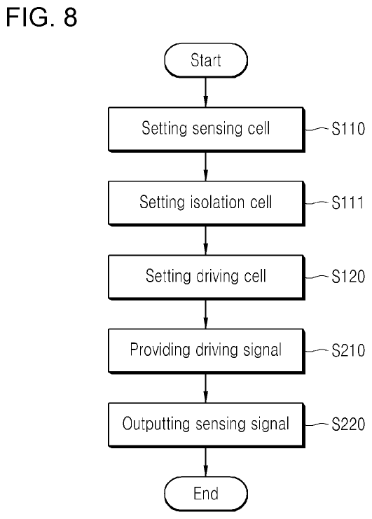

FIG. 5 is a top view illustrating a fingerprint sensor using a pseudo-direct method in accordance with another exemplary embodiment.

Referring to (a) of FIG. 5, the fingerprint sensor using the pseudo-direct method in accordance with another exemplary embodiment may set one cell disposed at a center of 5×5 areas as the sensing cell 110, eight cells disposed around the one central cell, which is set as the sensing cell 100, as the isolation cell 130, and the rest 16 cells as the driving cell 120.

Alternatively, the above-described sensing cell 110, driving cell 120, and isolation cell 130 may be provided as in (b) of FIG. 5.

For example, in 7×7 areas, one cell disposed at a center may be set as the sensing cell 110, cells in two rows and two columns, which are adjacent to the sensing cell 110, may be set as the isolation cell 130, and the rest 24 cells may be set as the driving cell 120.

In the above-described arrangement, as two rows of isolation cells 130 are provided between the sensing cell 110 and the driving cell 120, a driving signal, which is directly applied from the driving cell 120 to the sensing cell 110, may be less than that of the configuration in (a) of FIG. 5.

Alternatively, the above-described sensing cell 110, driving cell 120, and isolation cell 130 may be provided as in (c) of FIG. 5.

For example, in 9×9 areas, three cells disposed at a center may be set as the sensing cell 110, cells in two rows and two columns, which are adjacent to the sensing cell 110, may be set as the isolation cell 130, and the rest cells may be set as the driving cell 120.

In the above-described cell arrangement, as a range of the sensing cell 110 increases, a fingerprint image in a wide area may be acquired at once. Also, as a distance between the sensing cell 110 and the driving cell 120 is wider than that of the configuration in (a) of FIG. 5, an error caused by the adjacent driving cell 120 may be reduced.

Also, a method for setting the plurality of cells of the sensor plate 110, 120, and 130 as the sensing cell 110, the driving cell 120, and the isolation cell 130 will be described below.

When the driving signal control switch 600 connected to an arbitrary cell of the sensor plate is in an on-state, and the isolation cell control switch 800 is in an off-state, as a driving signal generated from the driving signal generation unit 300 is provided to the cell, the cell may operate as the driving cell 120, a cell in which all of the driving signal control switch 600 and the isolation cell control switch 800 are in an off-state may operate as the sensing cell 110, and a cell in which the driving signal control switch 600 is in an off-state, and the isolation cell control switch 800 is in an on-state may operate as the isolation cell 130.

When the sensor plate is set as described above, as the isolation cell 130 provided between the sensing cell 110 and the driving cell 120 serves like the ground, a driving signal, which is applied to the sensing cell 110 without passing the finger 200, of driving signals applied from the driving cell 120 to the finger 200 may be remarkably reduced to improve sensitivity of fingerprint recognition.

Also, the active output voltage feedback unit 400 may include: a buffer circuit part that feedbacks an output terminal of an operational amplifier to an inverting input terminal of the operational amplifier; a first switch and a second switch, which are parallel-connected to the other end of the driving signal control switch; a storage capacitor having one end connected between one end of the first switch and a non-inverting input terminal of the operational amplifier of the buffer circuit part and the other end connected to the ground; and a reset switch having one end connected to the one end of the first switch, the non-inverting input terminal of the operational amplifier of the buffer circuit part, and one end of the storage capacitor and the other end connected to the ground.

More particularly, as the buffer circuit part operates as a voltage follower, a parasitic capacitor C_{shield} may be charged so that the input terminals of the buffer circuit have the same voltage as each other. Thus, the parasitic capacitor C_{shield} may be removed.

Also, the active output voltage feedback unit 400 may integrate a capacitance detected by the sensing cell 120 to the storage capacitor and output the integrated capacitance to the output terminal of the active output voltage feedback unit.

3. A method for sensing a fingerprint using a pseudo-direct method in accordance with an exemplary embodiment

FIG. 7 is a flowchart showing a method for sensing a fingerprint using a pseudo-direct method in accordance with an exemplary embodiment.

Referring to FIG. 7, a method for sensing a fingerprint using a pseudo-direct method in accordance with an exemplary embodiment includes: a process S110 of setting a sensing cell, which sets a portion of a plurality of cells of a sensor plate as a sensing cell; a process S120 of setting a driving cell, which sets cells, which are not set as the sensing cell, of the plurality of cells of the sensor plate as a driving cell; a process S210 of providing a driving signal, which provides a driving signal to the driving cell; and a process S220 of outputting a sensing signal, which integrates a capacitance detected in the sensing cell by a signal applied from the driving cell to a finger and outputs the integrated capacitance.

More particularly, the process S110 of setting a sensing cell may turn-off a driving signal control switch to set a portion of the plurality of cells of the sensor plate as the sensing cell, and the process S120 of setting a driving cell may turn-on the driving signal control switch to set cells, which are not set as the sensing cell, of the plurality of cells of the sensor plate as the driving cell.

Also, the driving cell, which is set in the process S120 of setting a driving cell, may be adjacent to the sensing cell, which is set in the process S110 of setting a sensing cell.

4. A method for sensing a fingerprint using a pseudo-direct method in accordance with another exemplary embodiment

FIG. 8 is a flowchart showing a method for sensing a fingerprint using a pseudo-direct method in accordance with another exemplary embodiment.

Referring to FIG. 8, a method for sensing a fingerprint using a pseudo-direct method in accordance with another exemplary embodiment includes: a process S110 of setting a sensing cell, which sets a portion of a plurality of cells of a sensor plate as a sensing cell; a process of setting an isolation cell, which sets a portion of cells disposed adjacent to the set sensing cells of the plurality of cells of the sensor plate as an isolation cell; a process S120 of setting a driving cell, which sets cells, which are not set as the sensing cell and the isolation cell, of the plurality of cells of the sensor plate as a driving cell; a process S210 of providing a driving signal, which provides a driving signal to the driving cell; and a process S220 of outputting a sensing signal, which integrates a capacitance detected in the sensing cell by a signal applied from the driving cell to a finger and outputs the integrated capacitance.

More particularly, the process S110 of setting the sensing cell may turn-off all of a driving signal control switch and an isolation cell control switch to set a portion of the plurality of cells of the sensor plate as the sensing cell, the process S111 of setting the isolation cell may turn-off the driving signal control switch and turn-on the isolation cell control switch to set a portion of cells disposed adjacent to the set sensing cell of the plurality of cells of the sensing plate as the isolation cell, and the process S120 of setting the driving cell may turn-on the driving signal control switch and turn-off the isolation cell control switch to set cells, which are not set as the sensing cell and the isolation cell, of the plurality of cells of the sensor plate as the driving cell.

Also, the isolation cell, which is set in the process S111 of setting the isolation cell, may be disposed adjacent to the sensing cell set in the process S110 of setting the sensing cell, and the driving cell, which is set in the process S120 of setting the driving cell, may be disposed adjacent to the isolation cell set in the process S120 of setting the driving cell.

That is, the isolation cell, which is set in the process S111 of setting the isolation cell, may be provided between the driving cell and the sensing cell to decrease a phenomenon in which the driving signal is applied directly from the driving cell to the sensing cell.

As described above, the technical idea of the present invention has been specifically described with respect to the above embodiments, but it should be noted that the foregoing embodiments are provided only for illustration while not limiting the present invention. Various embodiments may be provided to allow those skilled in the art to understand the scope of the preset invention, but the present invention is not limited thereto.

What is claimed is:

1. A fingerprint sensor comprising:
a sensor plate comprising a plurality of cells;
a driving signal generation unit disposed between an external voltage input terminal and the sensor plate to generate a driving signal;
an active output voltage feedback unit disposed between one end of a variable capacitor and a voltage output terminal; and
a shielding plate provided below the sensor plate and connected to an output terminal of the active output voltage feedback unit to block a noise generated from a circuit disposed below the sensor plate,
wherein the fingerprint sensor further comprises a driving signal control switch having one end connected to the driving signal generation unit and the other end connected to each of the plurality of cells of the sensor plate to set at least a portion of the plurality of cells of the sensor plate as a sensing cell and at least a portion of cells disposed around the set sensing cells as a driving cell,
wherein the sensing cell measures a capacitance formed between a finger and the sensing plate, and the driving cell provides a driving signal generated from the driving signal generation unit to the finger.

2. The fingerprint sensor of claim 1, wherein the plurality of cells of the sensor plate are set: as the driving cell when the driving signal control switch is in an on-state; and as the sensing cell when the driving signal control switch is in an off-state, and
the driving cell is disposed adjacent to the sensing cell.

3. A fingerprint sensor comprising:
a sensor plate comprising a plurality of cells;
a driving signal generation unit disposed between an external voltage input terminal and the sensor plate to generate a driving signal;
an active output voltage feedback unit disposed between one end of a variable capacitor and a voltage output terminal; and
a shielding plate provided below the sensor plate and connected to an output terminal of the active output voltage feedback unit to block a noise generated from a circuit disposed below the sensor plate,
wherein the fingerprint sensor further comprises: a driving signal control switch having one end connected to the driving signal generation unit and the other end connected to the sensor plate to set at least a portion of the plurality of cells of the sensor plate as a sensing cell and at least a portion of cells, which are spaced by a predetermined area from the set sensing cells, as a driving cell; and
an isolation cell control switch having one end connected to the driving signal control switch and the other end connected to the ground to set cells in a predetermined area, in which the sensing cell and the driving cell are spaced apart from each other, as an isolation cell,
wherein the sensing cell measures a capacitance formed between a finger and the sensing plate, the driving cell provides a driving signal generated from the driving signal generation unit to the finger, and the isolation cell is connected to the ground to remove a capacitance directly applied from the driving cell to the sensing cell.

4. The fingerprint sensor of claim 3, wherein the plurality of cells of the sensor plate operate: as the driving cell when the driving signal control switch is in a on-state, and the isolation cell control switch is in an off-state; as the sensing cell when all of the driving signal control switch and the isolation cell control switch are in the off-state; and as the isolation cell when the driving signal control switch is in the off-state, and the isolation cell control switch is in an on-state, and
the isolation cell is provided between the driving cell and the sensing cell.

5. The fingerprint sensor of claim 1, wherein the active output voltage feedback unit comprises:
a buffer circuit part configured to feedback an output terminal of an operational amplifier to an inverting input terminal of the operational amplifier;
a first switch and a second switch, which are parallel-connected to the other end of the driving signal control switch and an isolation cell control switch, respectively;
a storage capacitor having one end connected between one end of the first switch and a non-inverting input terminal of the operational amplifier of the buffer circuit part and the other end connected to the ground; and
a reset switch having one end connected to the one end of the first switch, the non-inverting input terminal of the operational amplifier of the buffer circuit part, and one end of the storage capacitor and the other end connected to the ground.

6. A method for sensing a fingerprint, the method comprising:
setting at least a portion of a plurality of cells of a sensor plate as a sensing cell configured to measure a capacitance formed between a finger and the sensing plate;
setting at least a portion of cells around the set sensing cells of the plurality of cells of the sensor plate as a driving cell configured to provide a driving signal to a finger;
providing a driving signal from the driving cell to the finger; and
outputting a sensing signal by integrating a capacitance detected in the sensing cell by the signal applied from the driving cell to the finger.

7. The method of claim 6, wherein the setting of the sensing cell turns-off a driving signal control switch to set a portion of the plurality of cells of the sensor plate as the sensing cell, and
the setting of the driving cell turns-on the driving signal control switch to set cells, which are not set as the sensing cell, of the cells of the sensor plate as the driving cell.

8. A method for sensing a fingerprint, the method comprising:
setting at least a portion of a plurality of cells of a sensor plate as a sensing cell configured to measure a capacitance formed between a finger and the sensing plate;
setting at least a portion of cells spaced by a predetermined area from the set sensing cells as a driving cell configured to provide a driving signal to a finger;
setting cells in a predetermined area in which the sensing cell and the driving cell are spaced apart from each other as an isolation cell;
providing a driving signal to the driving cell; and
outputting a sensing signal by integrating a capacitance detected in the sensing cell by the signal applied from the driving cell to the finger.

9. The method of claim 8, the setting of the sensing cell turns-off all of a driving signal control switch and an isolation cell control switch to set a portion of the plurality of cells of the sensor plate as the sensing cell,
the setting of the isolation cell turns-off the driving signal control switch and turns-on the isolation cell control switch to set a portion of cells, which are adjacent to the set sensing cells of the plurality of cells of the sensor plate, as the isolation cell, and
the setting of the driving cell turns-on the driving signal control switch and turns-off the isolation cell control switch to set cells, which are not set as the sensing cell and the isolation cell, of the plurality of cells of the sensor plate as the driving cell.
